SEQUENCE LISTING (38 sequences)

SEQ ID NO: 1 - protein, 13 aa, Artificial Sequence
  Synthetic Construct
KASGYTFTDYYIN

SEQ ID NO: 2 - protein, 17 aa, Artificial Sequence
  Synthetic Construct
WINPGSGNTKYNEKFKG

SEQ ID NO: 3 - protein, 8 aa, Artificial Sequence
  Synthetic Construct
TREGETVY

SEQ ID NO: 4 - protein, 16 aa, Artificial Sequence
  Synthetic Construct
KSSQSLLYSRGKTYLN

SEQ ID NO: 5 - protein, 8 aa, Artificial Sequence
  Synthetic Construct
YAVSKLDS

SEQ ID NO: 6 - protein, 8 aa, Artificial Sequence
  Synthetic Construct
YDVSKLDS

SEQ ID NO: 7 - protein, 9 aa, Artificial Sequence
  Synthetic Construct
VQGTHYPFT

SEQ ID NO: 8 - protein, 115 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSS

SEQ ID NO: 9 - protein, 112 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIK

SEQ ID NO: 10 - protein, 112 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIK

SEQ ID NO: 11 - protein, 444 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCTREGETVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 219 aa, Artificial Sequence
  Synthetic Construct
DVVMTQSPLSLPVTLGQPASISCKSSQSLLYSRGKTYLNWFQQRPGQSPRRLIYDVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 219 aa, Artificial Sequence
  Synthetic Construct
DIQMTQSPSTLSASVGDRVTITCKSSQSLLYSRGKTYLNWLQQKPGKAPKLLIYAVSKLDSGVPSRFSGSGSGTEFTLTISSLQPDDFATYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - DNA, 1332 bp, Artificial Sequence
  Synthetic Construct
caggtgcagctggtgcagtctggggctgaggtgaagaagcctgggtcctcggtgaaggtctcctgcaaggcttctggatacaccttcaccgactattatatcaactgggtgcgacaggcccctggacaagggcttgagtggatgggatggatcaaccctggcagtggtaatacaaagtacaatgagaagttcaagggcagagtcacgattaccgcggacgaatccacgagcacagcctacatggagctgagcagcctgagatctgaggacacggccgtgtattactgtacaagagaaggcgagacggtctactggggccagggaaccctggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 15 - DNA, 657 bp, Artificial Sequence
  Synthetic Construct
gatgttgtgatgactcagtctccactctccctgcccgtcacccttggacagccggcctccatctcctgcaagtctagtcaaagcctcctgtacagtcgcggaaaaacctacttgaattggtttcagcagaggccaggccaatctccaaggcgcctaatttatgatgtttctaaactggactctggggtcccagacagattcagcggcagtgggtcaggcactgatttcacactgaaaatcagcagggtggaggctgaggatgttggggtttattactgcgtgcaaggtacacactaccctttcacttttggccaagggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 16 - DNA, 657 bp, Artificial Sequence
  Synthetic Construct
gacatccagatgacccagtctccttccaccctgtctgcatctgtaggagacagagtcaccatcacttgcaagtccagtcagagtctcctgtacagtcgcggaaaaacctatttgaactggctccagcagaaaccagggaaagcccctaagctcctgatctatgctgtctccaaactggacagtggggtcccatcaaggttcagcggcagtggatctgggacagaattcactctcaccatcagcagcctgcagcctgatgattttgcaacttattactgcgtgcagggtacacattatcctttcacttttggccaggggaccaagctggagatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 17 - protein, 16 aa, Artificial Sequence
  Synthetic Construct
KSSQSLLYSRGKTYLN

SEQ ID NO: 18 - protein, 7 aa, Artificial Sequence
  Synthetic Construct
AVSKLDS

SEQ ID NO: 19 - protein, 9 aa, Artificial Sequence
  Synthetic Construct
VQGTHYPFT

SEQ ID NO: 20 - protein, 10 aa, Artificial Sequence
  Synthetic Construct
GYDFTRYYIN

SEQ ID NO: 21 - protein, 10 aa, Artificial Sequence
  Synthetic Construct
GYTFTRYYIN

SEQ ID NO: 22 - protein, 17 aa, Artificial Sequence
  Synthetic Construct
WINPGSGNTKYNEKFKG

SEQ ID NO: 23 - protein, 6 aa, Artificial Sequence
  Synthetic Construct
EGITVY

SEQ ID NO: 24 - protein, 6 aa, Artificial Sequence
  Synthetic Construct
EGTTVY

SEQ ID NO: 25 - protein, 112 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 26 - protein, 115 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 27 - protein, 115 aa, Artificial Sequence
  Synthetic Construct
QVQLVQSGAEVKKPGSSVKVSCKASGYTFTRYYINWVRQAPGQGLEWMGWINPGSGNTKYNEKFKGRVTITADESTSTAYMELSSLRSEDTAVYYCAREGTTVYWGQGTTVTVSS

SEQ ID NO: 28 - protein, 219 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 444 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 444 aa, Artificial Sequence
  Synthetic Construct
QVQLVQSGAEVKKPGSSVKVSCKASGYTFTRYYINWVRQAPGQGLEWMGWINPGSGNTKYNEKFKGRVTITADESTSTAYMELSSLRSEDTAVYYCAREGTTVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 40 aa, Artificial Sequence
  Synthetic Construct
  MISC_FEATURE (1)..(1): Xaa in position 1 = pyroglutamic acid
XFRHDSGYEVHHQKLVFFAEDVGSNKGAIIGLMVGGVVIA

SEQ ID NO: 32 - protein, 112 aa, Artificial Sequence; identical to SEQ ID NO: 25
  Synthetic Construct

SEQ ID NO: 33 - protein, 219 aa, Artificial Sequence; identical to SEQ ID NO: 28
  Synthetic Construct

SEQ ID NO: 34 - protein, 115 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSS

SEQ ID NO: 35 - protein, 444 aa, Artificial Sequence
  Synthetic Construct
QVQLVQSGAEVKKPGSSVKVSCKASGYTFTDYYINWVRQAPGQGLEWMGWINPGSGNTKYNEKFKGRVTITADESTSTAYMELSSLRSEDTAVYYCAREGETVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 10 aa, Artificial Sequence
  Synthetic Construct
GYTFTDYYIN

SEQ ID NO: 37 - protein, 6 aa, Artificial Sequence
  Synthetic Construct
EGETVY

SEQ ID NO: 38 - protein, 42 aa, Artificial Sequence [sequence omitted]

(19) United States
(12) Patent Application Publication
(10) Pub. No.: US 2019/0038613 A1
(43) Pub. Date: Feb. 7, 2019

(54) COMBINATION THERAPY

(71) Applicant: Eli Lilly and Company, Indianapolis, IN (US)

(72) Inventors: Ronald Bradley DeMattos, Zionsville, IN (US); Patrick Cornelius May, Fort Wayne, IN (US); John Randall Sims, II, Zionsville, IN (US)

(21) Appl. No.: 16/076,889

(22) PCT Filed: Mar. 10, 2017
(86) PCT No.: PCT/US17/21753

§ 371 Date: Aug. 9, 2018

Related U.S. Application Data
(60) Provisional application No. 62/308,369, filed on Mar. 15, 2016.

(51) Int. Cl. A61K 31/4439; A61P 25/28; A61K 39/395

(52) U.S. Cl. CPC A61K 31/4439; A61K 2300/00; A61K 39/3955; A61P 25/28

(57) ABSTRACT

The present invention provides a method of treating a cognitive or neurodegenerative disease, comprising administering to a patient in need of such treatment an effective amount of (1r, 1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof); in combination with an effective amount of an anti-N3pGlu Abeta antibody.

The present invention relates to a combination of a BACE inhibitor with an anti-N3pGlu Abeta antibody, and to methods of using the same to treat diseases characterized by deposition of amyloid β (Abeta or Aβ) peptide, such as Alzheimer's disease (AD).

Alzheimer's disease is a devastating neurodegenerative disorder that affects millions of patients worldwide. In view of the currently approved agents on the market which afford only transient, symptomatic benefits to the patient, there is a significant unmet need in the treatment of Alzheimer's disease. Alzheimer's disease is characterized by the generation, aggregation, and deposition of Abeta in the brain. Complete or partial inhibition of beta-secretase (beta-site amyloid precursor protein-cleaving enzyme; BACE) has been shown to have a significant effect on plaque-related and plaque-dependent pathologies in mouse models. This suggests that even small reductions in Abeta peptide levels might result in a long-term significant reduction in plaque burden and synaptic deficits, thus providing significant therapeutic benefits, particularly in the treatment of Alzheimer's disease.

Moreover, antibodies that specifically target N3pGlu Abeta have been shown to lower plaque level in vivo (U.S. Patent Application Publication No. 2013/0142806). These antibodies are referred to herein as “anti-N3pGlu Abeta”. N3pGlu Abeta, also referred to as N3pGlu Aβ, N3pE or A beta_{p3 . . . 42}, is a truncated form of the Abeta peptide found only in plaques. Although N3pGlu Abeta peptide is a minor component of the deposited Abeta in the brain, studies have demonstrated that N3pGlu Abeta peptide has aggressive aggregation properties and accumulates early in the deposition cascade.

A combination of a BACE inhibitor with an antibody that binds N3pGlu Abeta peptide is desired to provide treatment for Abeta peptide-mediated disorders, such as Alzheimer's disease, which may be more effective than either drug alone. For example, treatment with such combination may allow for use of lower doses of either or both drugs as compared to each drug used alone, potentially leading to lower side effects (or a shorter duration of one or the other therapy) while maintaining efficacy. It is believed that targeting the removal of deposited forms of Abeta with an N3pG antibody and a BACE inhibitor will facilitate the phagocytic removal of pre-existing plaque deposits while at the same time reduce or prevent further deposition of Abeta by inhibiting the generation of Abeta.

U.S. Pat. No. 8,415,483 discloses molecules which possess BACE inhibitory activity and are further disclosed as useful therapeutic agents for neurodegenerative disease caused by Aβ peptide, such as Alzheimer's type dementia. U.S. Patent Application Publication No. 2014/0031379 entitled “Camsylate Salt” provides a camslate salt of one of the compounds of U.S. Pat. No. 8,415,483. U.S. Pat. No. 8,278,334 discloses a method of treating a cognitive or neurodegenerative disease comprising administering a substituted cyclic amine BACE-1 inhibitor with an anti-amyloid antibody. Further, J. Neuroscience, 34(35), pages 11621-11630 (2014) discloses that combined treatment with a BACE inhibitor and an anti-A beta antibody Gentenerumab enhances amyloid reduction in APP_{London} mice. In addition, R. DeMattos, et. al., disclosed at the 2015 Alzheimer's Association International Conference (July 18-23; Abstract ID No. 6350) an investigation of dose-responses and longitudinal effects of combination therapy with a plaque specific Abeta antibody (N3pG) and BACE inhibitor in aged PDAPP transgenic mice.

Accordingly, the present invention provides a method of treating a cognitive or neurodegenerative disease, comprising administering to a patient in need of such treatment an effective amount of a camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine; in combination with an effective amount of an anti-N3pGlu Abeta antibody. The camsylate salt of (1r,1′R,4R)-4-methoxy-5″-methyl-6′-[5-(prop-1-yn-1-yl)pyridin-3-yl]-3″H-dispiro[cyclohexane-1,2′-indene-1′,2″-imidazol]-4″-amine is outlined and disclosed (including methods of making this and other compounds in U.S. Patent Application Publication No. 2014/0031379 entitled “Camsylate Salt”).

The present invention also provides a method of treating a cognitive or neurodegenerative disease or a disease that is characterized by the deposition of Abeta, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine or a pharmaceutically acceptable salt thereof (such as, for example, the camsylate salt); in combination with an effective amount of an anti-N3pGlu Abeta antibody. (The compound of (1r,1′R,4R)-4-methoxy-5″-methyl-6′-[5-(prop-1 -yn-1-yl)pyridin-3-yl]-3′H-dispiro[cyclohexane-1,2′-indene-1′,2″-imidazol]-4″-amine is outlined and disclosed (including methods of making this and other compounds) in U.S. Pat. No. 8,415,483 entitled “Compounds and Their Use as BACE Inhibitors”).

The present invention also provides a method of treating a disease that is characterized by the deposition of A beta, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of treating clinical or pre-clinical Alzheimer's disease, Down's syndrome, and clinical or pre-clinical CAA comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention also provides a method of treating prodromal AD (sometimes also referred to as Aβ-related mild cognitive impairment, or MCI), mild AD, moderate AD and severe AD, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of treating prodromal AD, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of treating mild AD, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of treating moderate AD, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), combination with an effective amount of an anti-N3pGlu Abeta antibody.

The present invention further provides a method of treating severe AD, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of slowing cognitive decline in a patient diagnosed with pre-clinical Alzheimer's disease or clinical Alzheimer's disease, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of slowing functional decline in a patient diagnosed with pre-clinical Alzheimer's disease or clinical Alzheimer's disease, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of reducing brain Aβ amyloid plaque load in a patient in diagnosed with pre-clinical Alzheimer's disease or clinical Alzheimer's disease, comprising administering an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further invention provides a method of preventing memory loss or cognitive decline in asymptomatic patients with low levels of Aβ1-42 in the cerebrospinal fluid (CSF) or Aβ plaques in the brain, comprising administering an effective amount of a compound which is (1r,1′R,4R)-4-methoxy-5″-methyl-6′-[5-(prop1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

In another embodiment the present invention provides a method of treating asymptomatic patients known to have an Alzheimer's disease-causing genetic mutation, comprising administering an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

Another embodiment the present invention provides a method for the prevention of the progression of mild cognitive impairment to Alzheimer's disease, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), in combination with an effective amount of an anti-N3pGlu Abeta antibody.

The present invention further provides a method of treating cerebral amyloid angiopathy (CAA), comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof) combination with an effective amount of an anti-N3pGlu Abeta antibody.

The present embodiments also provide a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), for use in simultaneous, separate, or sequential combination with an anti-N3pGlu Abeta antibody, for use in therapy.

Another embodiment provides a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), for use in simultaneous, separate, or sequential combination with an anti-N3pGlu Abeta antibody, for use in the treatment of a disease characterized by deposition of Aβ. In another embodiment of the present invention provides a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), for use in simultaneous, separate, or sequential combination with an anti-N3pGlu Abeta antibody, for use in treatment of clinical or pre-clinical Alzheimer's disease, Down's syndrome, and clinical or pre-clinical cerebral amyloid angiopathy

The invention further provides a pharmaceutical composition comprising a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), with one or more pharmaceutically acceptable carriers, diluents, or excipients, in combination with a pharmaceutical composition of an anti-N3pGlu Abeta antibody, with one or more pharmaceutically acceptable carriers, diluents, or excipients.

In addition, the invention provides a kit, comprising a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), and an anti-N3pGlu Abeta antibody. The invention further provides a kit, comprising a pharmaceutical composition, comprising a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), with one or more pharmaceutically acceptable carriers, diluents, or excipients, and a pharmaceutical composition, comprising an anti-N3pGlu Abeta antibody with one or more pharmaceutically acceptable carriers, diluents, or excipients. As used herein, a “kit” includes separate containers of each component, wherein one component is a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), and another component is an anti-N3pGlu Abeta antibody, in a single package. A “kit” may also include separate containers of each component, wherein one component is a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), and another component is an anti-N3pGlu Abeta antibody, in separate packages with instructions to administer each component as a combination.

The invention further provides the use of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), for the manufacture of a medicament for the treatment of Alzheimer's disease, mild Alzheimer's disease, prodromal Alzheimer's disease or for the prevention of the progression of mild cognitive impairment to Alzheimer's disease wherein the medicament is to be administered simultaneously, separately or sequentially with an anti-N3pGlu Abeta antibody.

In an embodiment of the present invention, the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ II). NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1. is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ NO: 7. HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

In other embodiments, the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ II) NO: 34;
d) LCVR of SEQ ID NO: 9 and HCVR of SEQ ID NO: 8; and
e) LCVR of SEQ ID NO: 10 and HCVR of SEQ ID NO: 8.

In further embodiments, the anti-N3pGlu Abeta antibody comprises a light chain (LC) and a heavy chain (HC), wherein said LC and HC are selected from the group consisting of
a) LC of SEQ ID NO: 28 and HC of SEQ ID NO: 29;
b) LC of SEQ H) NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

In other embodiments, the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

In some embodiments, the anti-N3pGlu Abeta antibody comprises Antibody I, which has a light chain (LC) and a heavy chain (HC) of SEQ ID NOs: 12 and 11 respectively. Antibody I further has a light chain variable region (LCVR) and a heavy chain variable region (HCVR) of SEQ ID NOs: 9 and 8 respectively. The HCVR of Antibody I further comprises HCDR1 of SEQ ID NO: 1, HCDR2 of SEQ ID NO: 2, and HCDR3 of SEQ ID NO: 3. The LCVR of Antibody I further comprises LCDR1 of SEQ ID NO: 4, LCDR2 of SEQ ID NO: 6 and LCDR3 of SEQ ID NO: 7 respectively.

In some embodiments, the anti-N3pGlu Abeta antibody comprises Antibody II, which has a light chain (LC) and a heavy chain (HC) of SEQ ID NOs: 13 and 11 respectively. Antibody II further has a light chain variable region (LCVR) and a heavy chain variable region (HCVR) of SEQ ID NOs: 10 and 8 respectively. The HCVR of Antibody II further comprises HCDR1 of SEQ ID NO: 1, HCDR2 of SEQ ID NO: 2, and HCDR3 of SEQ NO: 3. The LCVR of Antibody II further comprises LCDR1 of SEQ ID NO: 4, LCDR2 of SEQ ID NO. 5, and LCDR3 of SEQ ID NO: 7 respectively.

In some embodiments, the anti-N3pGlu Abeta antibody comprises B12L, which has a light chain (LC) and a heavy chain (HC) of SEQ ID NOs: 28 and 29 respectively. B12L further has a light chain variable region (LCVR) and a heavy chain variable region (HCVR) of SEQ ID NOs: 25 and 26 respectively. The HCVR of B12L further comprises HCDR1 of SEQ ID NO: 20, HCDR2 of SEQ ID NO: 22 and HCDR3 of SEQ ID NO: 23. The LCVR of B12L further comprises LCDR1. of SEQ ID NO. 17. LCDR2 of SEQ NO: 18 and. LCDR3 of SEQ ID NO: 19 respectively.

In some embodiments, the anti-N3pGlu Abeta antibody comprises R17L which has a light chain (LC) and a heavy chain (HC) of SEQ ID NOs: 28 and 30 respectively. R17L further has a light chain variable region (LCVR) and a heavy chain variable region (HCVR) of SEQ ID NOs: 25 and 27 respectively. The HCVR of R17L further comprises HCDR1 of SEQ ID NO: 21, HCDR2 of SEQ ID NO: 22 and HCDR3 of SEQ ID NO: 24. The LCVR of R17L further comprises LCDR1 of SEQ ID NO. 17, LCDR2 of SEQ NO: 18 and LCDR3 of SEQ ID NO: 19 respectively.

In some embodiments, the anti-N3pGlu Abeta antibody comprises hE8L which has a light chain (LC) and a heavy chain (HC) of SEQ ID NOs: 33 and 35 respectively. hE8L further has a light chain variable region (LCVR) and a heavy chain variable region (HCVR) of in SEQ ID NOs: 32 and 34 respectively. The HCVR of hE8L further comprises HCDR1 of SEQ ID NO: 36, HCDR2 of SEQ ID NO: 22 and HCDR3 of SEQ ID NO: 37. The LCVR of hE8L further comprises LCDR1 of SEQ ID NO. 17, LCDR2 of SEQ ID NO. 18 and LCDR3 of SEQ ID NO: 19 respectively.

For purposes of clarity, the molecule (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine refers to the following structure:

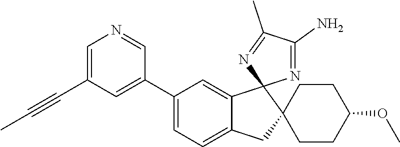 [SMILES: CC#Cc1cncc(-c2ccc3c(c2)[C@@]2(N=C(C)C(N)=N2)[C@]2(CC[C@H](OC)CC2)C3)c1]

One method of making such molecule is disclosed in U.S. Pat. No. 8,415,483. (See, for example, the synthesis associated with Example 20a of U.S. Pat. No. 8,415,483). Further, the camsylate salt of this molecule can exist in either of the following forms:

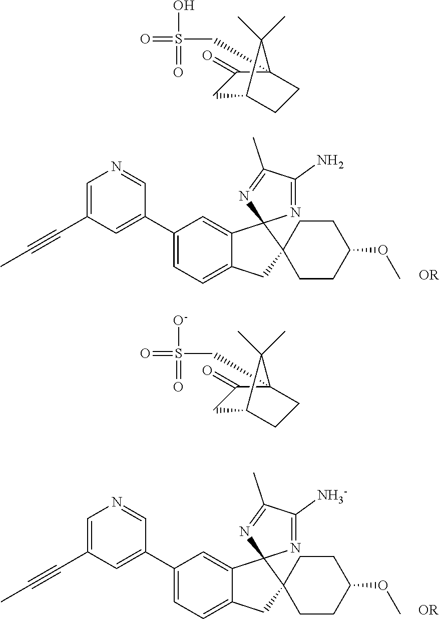

One method of making such molecule is disclosed in U.S. Patent Application Publication No. 2014/0031379.

One of ordinary skill in the art will appreciate and recognize that “anti-N3pGlu Abeta antibody”, and the specific antibodies, “B12L” and “R17L” are identified and disclosed along with methods for making and using said antibodies by one of ordinary skill in the art, in U.S. Pat. No. 8,679,498 B2, entitled “Anti-N3pGlu Amyloid Beta Peptide Antibodies and Uses Thereof”, issued Mar. 25, 2014 (U.S. Ser. No. 13/810,895). See for example Table 1 of U.S. Pat. No. 8,679,498 B2. Each of these two antibodies (e.g., “B12L” and “R17L”) may be used as the anti-N3pGlu Abeta antibody of the present invention. In other embodiments, the anti-N3pGlu Abeta antibody may comprise the antibody “hE8L” described herein. In further embodiments, the anti-N3pGlu Abeta antibody may comprise “Antibody I” outlined herein. In yet further embodiments, the anti-N3pGlu Abeta antibody may comprise “Antibody II” outlined herein.

In addition, amino acid sequences for certain antibodies used in the present invention are provided below in Table A:

TABLE A
Antibody SEQ ID NOs

| | Light | Heavy | | |
|---|---|---|---|---|
| Antibody | Chain | Chain | LCVR | HCVR |
| B12L | 28 | 29 | 25 | 26 |
| R17L | 28 | 30 | 25 | 27 |
| hE8L | 33 | 35 | 32 | 34 |
| Antibody I | 12 | 11 | 9 | 8 |
| Antibody II | 13 | 11 | 10 | 8 |

With respect to “Antibody I” and “Antibody II”, additional amino acid sequences for such antibodies are provided in Table B:

TABLE B
Additional SEQ ID NOs For Claimed Antibodies
Antibody SEQ ID NOs

| Antibody | LCDR1 | LCDR2 | LCDR3 |
|---|---|---|---|
| B12L | 17 | 18 | 19 |
| R17L | 17 | 18 | 19 |
| hE8L | 17 | 18 | 19 |
| Antibody I | 4 | 6 | 7 |
| Antibody II | 4 | 5 | 7 |

Antibody SEQ ID NOs

| Antibody | HCDR1 | HCDR2 | HCDR3 |
|---|---|---|---|
| B12L | 20 | 22 | 23 |
| R17L | 21 | 22 | 24 |
| hE8L | 36 | 22 | 37 |
| Antibody I | 1 | 2 | 3 |
| Antibody II | 1 | 2 | 3 |

The antibodies of the present invention bind to N3pGlu Aβ. The sequence of N3pGlu Aβ is the amino acid sequence of SEQ ID NO: 31. The sequence of Aβ is SEQ ID NO: 38.

As used herein, an “antibody” is an immunoglobulin molecule comprising two Heavy Chain (HC) and two Light Chain (LC) interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present invention is based on the well-known numbering conventions such as the following: Kabat, et al,, Ann. NY. Acad. Sci. 190:382-93 (1971); Kabat et al., Sequences of Proteins of Immunological Interest, Fifth Edition, U.S. Department of Health and Human Services, NIH Publication No. 91-3242 (1991); and North numbering convention (North et al., A New Clustering of Antibody CDR Loop Conformations, Journal of Molecular Biology, 406:228-256 (2011)).

As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

Following expression and secretion of the antibody, the medium is clarified to remove cells and the clarified media is purified using any of many commonly-used techniques. The purified antibody may be formulated into pharmaceutical compositions according to well-known methods for formulating proteins and antibodies for parenteral administration, particularly for subcutaneous, intrathecal, or intravenous administration. The antibody may be lyophilized, together with appropriate pharmaceutically-acceptable excipients, and then later reconstituted with a water-based diluent prior to use. Alternatively, the antibody may be formulated in an aqueous solution and stored prior to use. In either case, the stored form and the injected form of the pharmaceutical compositions of the antibody will contain a pharmaceutically-acceptable excipient or excipients, which are ingredients other than the antibody. Whether an ingredient is pharmaceutically-acceptable depends on its effect on the safety and effectiveness or on the safety, purity, and potency of the pharmaceutical composition. If an ingredient is judged to have a sufficiently unfavorable effect on safety or effectiveness (or on safety, purity, or potency) to warrant it not being used in a composition for administration to humans, then it is not pharmaceutically-acceptable to be used in a pharmaceutical composition of the antibody.

The term “disease characterized by deposition of Aβ,” is a disease that is pathologically characterized by All deposits in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes some form of brain plaque imagining, mental or cognitive assessment (e.g. Clinical Dementia Rating- summary of boxes (CDR-SB), Mini-Mental State Exam 25 (MMSE) or Alzheimer's Disease Assessment Scale-Cognitive (ADAS-Cog)) or functional assessment (e.g. Alzheimer's Disease Cooperative Study-Activities of Daily Living (ADCS-ADL). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease and severe Alzheimer's disease. The term “pre-clinical Alzheimer's disease” is a stage that precedes clinical Alzheimer's disease, where measurable changes in biomarkers (such as CSP Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable.

As used herein, the terms “treating”, “to treat”, or “treatment”, includes restraining, slowing, stopping, reducing, or reversing the progression or severity of an existing symptom, disorder, condition, or disease.

As used herein, the terra “patient” refers to a human.

The term “inhibition of production of Abeta peptide” is taken to mean decreasing of in vivo levels of A betapeptide in a patient.

The term “prevention” means prophylactic administration of the combination of the compounds outlined herein and the antibody to an asymptomatic patient or a patient with pre-clinical Alzheimer's disease to prevent progression of the disease.

As used herein, the term “effective amount” refers to the amount or dose of compound comprising (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), and to the amount or dose of an anti-N3pGlu Abeta antibody administered to the patient, that provides the desired effect in the patient under diagnosis or treatment. It is understood that the combination therapy of the present invention is carried out by administering a compound comprising (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (including the camsylate salt thereof), together with the anti-N3pGlu Abeta antibody in any manner which provides effective levels of the compound (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof and the anti-N3pGlu Abeta antibody in the body.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compounds of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt (such as, for example, the camsylate salt) thereof are generally effective over a wide dosage range in the combination of the present invention. For example, dosages of the compound per day normally fall within the range of about 0.1 mg/day to about 1000 mg/day, preferably about 0.1 mg/day to about 500 mg/day, and most preferably about 0.1 mg/day to about 100 mg/day. In some embodiments, the dose of the molecule is 20 mg or 50 mg. In addition, the anti-N3pGlu Abeta antibody is generally effective over a wide dosage range in the combination of the present invention. In some instances dosage levels below the lower limit of the aforesaid ranges may be more than adequate, while in other cases still larger doses may be employed with acceptable adverse events, and therefore the above dosage range is not intended to limit the scope of the invention in any way.

The BACE inhibitors and the antibodies of the present invention are preferably formulated as pharmaceutical compositions administered by any route which makes the compound bioavailable. The route of administration may be varied in any way, limited by the physical properties of the drugs and the convenience of the patient and the caregiver. Preferably, anti-N3pGlu Abeta antibody compositions are for parenteral administration, such as intravenous or subcutaneous administration. In addition, the BACE inhibitor, such as the compound of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine I, or pharmaceutically acceptable salt thereof, is for oral, parenteral, or transdermal administration, including intravenous or subcutaneous administration. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006).

As used herein, the phrase “in combination with” refers to the administration of the BACE inhibitor, such as a compound of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof (such as, for example, the camsylate salt), with an anti-N3pGlu Abeta antibody, such as an anti-N3pGlu Abeta antibody simultaneously, or sequentially in any order, or any combination thereof. The two molecules may be administered either as part of the same pharmaceutical composition or in separate pharmaceutical compositions. The BACE inhibitor can be administered prior to, at the same time as, or subsequent to administration of the anti-N3pGlu Abeta antibody, or in some combination thereof. Where the anti-N3pGlu Abeta antibody is administered at repeated intervals (e.g. during a standard course of treatment), the BACE inhibitor can be administered prior to, at the same time as, or subsequent to, each administration of the anti-N3pGlu Abeta antibody, or some combination thereof, or at different intervals in relation to therapy with the anti-N3pGlu Abeta antibody, or in a single or series of dose(s) prior to, at any time during, or subsequent to the course of treatment with the anti-N3pGlu Abeta antibody.

As used herein, “BSA” refers to Bovine Serum Albumin; “EDTA” refers to ethylenediaminetetraacetic acid; “ee” refers to enantiomeric excess; “Ex” refers to example; “F12” refers to Ham's F12 medium; “hr refers to hour or hours; “HRP” refers to Horseradish Peroxidase; “IC₅₀” refers to the concentration of an agent that produces 50% of the maximal inhibitory response possible for that agent; “min” refers to minute or minutes; “PBS” refers to Phosphate Buffered Saline; “PDAPP” refers to platelet derived amyloid precursor protein; “Prep” refers to preparation; “psi” refers to pounds per square inch; “Rₜ” refers to retention time; “SCX” refers to strong cation exchange chromatography; “THF” refers to tetrahydrofuran and “TMB” refers to 3,3′,5,5′-teramethylbenzidine.

### EXPRESSION AND PURIFICATION OF ENGINEERED N3PGLU Aβ ANTIBODIES

Anti-N3pGlu Aβ antibodies (for example, Antibody I or II) of the present invention can be expressed and purified essentially as follows. A glutamine synthetase (GS) expression vector containing the DNA sequence encoding the LC amino acid sequence of SEQ ID NO: 12 or 13 and the DNA sequence encoding the HC amino acid sequence of SEQ ID NO: 11 is used to transfect a Chinese hamster ovary cell line (CHO) by electroporation. The expression vector encodes an SV Early (Simian Virus 40E) promoter and the gene for GS. Post-transfection, cells undergo bulk selection with 0-50 μM L-methionine sulfoximine (MSX). Selected bulk cells or master wells are then scaled up in serum-free, suspension cultures to be used for production.

Clarified medium, into which the antibody has been secreted, is applied to a Protein A affinity column that has been equilibrated with a compatible buffer, such as phosphate buffered saline (pH 7.4). The column is washed with 1 M NaCl to remove nonspecific binding components. The bound anti-N3pGlu Aβ antibody is eluted, for example, with sodium citrate at pH (approx.) 3.5 and fractions are neutralized with 1 M Tris buffer. Anti-N3pGlu Aβ antibody fractions are detected, such as by SDS-PAGE or analytical size-exclusion, and then are pooled. Anti-N3pGlu Aβ antibody (Antibody I or Antibody II) of the present invention is concentrated in either PBS buffer at pH 7.4 or 10 mM NaCitrate buffer, 150 mM NaCl at pH around 6. The final material can be sterile filtered using common techniques. The purity of the anti-N3pGlu Aβ antibody is greater than 95%. The anti-N3pGlu Aβ antibody (Antibody I or Antibody II) of the present invention may be immediately frozen at −70° C. or stored at 4° C. for several months.

### Binding Affinity and Kinetics

The binding affinity and kinetics of an anti-N3pGlu Aβ antibody (Antibody I or Antibody II) to pE3-42 Aβ peptide or to Aβ 1-40 peptide is measured by surface plasmon resonance using BIACORE® 3000 (GE Healthcare). The binding affinity is measured by capturing the anti-N3pGlu Aβ antibody via immobilized protein A on a BIACORE® CMS chip, and flowing pE3-42 Aβ peptide or Aβ 1-40 peptide, starting from 100 nM in 2-fold serial dilution down to 3.125 nM. The experiments are carried out at 25° C. in HBS-EP buffer (GE Healthcare BR100669; 10 mM HEPES, 150 mM NaCl, 3 mM EDTA, 0.05% surfactant P20, pH 7.4).

For each cycle, the antibody is captured with 5 μL injection of antibody solution at a 10 μg/mL concentration with 10 μL/min, flow rate. The peptide is bound with 250 μL injection at 50 μL/min, and then dissociated for 10 minutes. The chip surface is regenerated with 5 μL injection of glycine buffer at pH 1.5 at 10 μL/mL flow rate. The data is fit to a 1:1 Langmiur binding model to derive kₒₙ, k_{off}, and to calculate K_{D}. Following procedures essentially as described above, the following parameters (shown in Table 2) were observed.

TABLE 2
Binding affinity and kinetics.

| Antibody | kₒₙ (×10⁵ 1/MS) | k_{off} (×10⁻⁴ 1/s) | K_{D} (nM) |
|---|---|---|---|
| I | 1.39 | 1.31 | 0.71 |
| II | 3.63 | 1.28 | 0.35 |

No appreciable binding to Aβ 1-40 was detected, indicating that Antibody I and Antibody II bound specifically to pE3-42 Aβ peptide as compared to Aβ 1-40.

### Ex Vivo Target Engagement

To determine ex vivo target engagement on brain sections from a fixed PDAPP brain, immunohistochemical analysis is performed with an exogenously added anti-N3pGlu Aβ antibody (Antibody I or Antibody II). Cryostat serial coronal sections from aged PDAPP mice (25-month old) are incubated with 20 μg/mL of an exemplified N3pGlu Aβ antibody of the present invention (Antibody I or Antibody II). Secondary HRP reagents specific for human IgG are employed and the deposited plaques are visualized with DAB-Plus (DAKO). Biotinylated murine 3D6 antibody followed by Step-HRP secondary is used as a positive control. The positive control antibody (biotinylated 3D6) labeled significant quantities of deposited Aβ in the PDAPP hippocampus, and the anti-N3pGlu Aβ antibodies (Antibody I or Antibody II) labeled a subset of deposits. These histological studies demonstrated that the anti-N3pGlu. Aβ antibodies (Antibody I and Antibody II) engaged deposited Aβ target ex vivo.

The following Examples and assays demonstrate how a study could be designed to verify (in animal models) that the combination of antibodies of the present invention, in combination with the compound outlined herein, may be useful for treating a disease characterized by deposition of Ail, such as of Alzheimer's disease, Downs syndrome, and CAA. It should be understood however, that the following descriptions are set forth by way of illustration and not limitation, and that various modifications may be made by one of ordinary skill in the art.

### Combination Study

### BACE Inhibitor Feeding Pilot Study

A pilot pharmacokinetic and pharmacodynamic study is performed in PDAPP mice fed a chow diet containing a BACE inhibitor, such as a compound described herein or pharmaceutically acceptable salt thereof in order to define doses that provide minimal to marked plasma and brain Abeta reduction by BACE inhibition alone. Young PDAPP mice are fed for 14 days a diet containing a chow diet containing the BACE inhibitor at “quasi-bid” equivalent doses of 3 mg/kg, 10 mg/kg, 30 mg/kg, or 100 mg/kg. The BACE inhibitor at ˜0.05, 0.15, 0.5, or 1.5 mg per gram of certified rodent diet #8728CM (Harlan labs) is mixed in a Sorvall mixer for 10 minutes and then mixed with Hobart mixer for 15 minutes prior to pelleting. Thirty-two young female PDAPP mice are randomized by parental line into 4 groups of 8 consisting of a vehicle-treatment group and the three doses of BACE inhibitor. Mice are allowed ad libitum access to food for 14 days and subsequently sacrificed. Mice are anesthetized with CO₂ and blood collected by cardiac puncture into EDTA-coated microcentrifuge tubes and stored on ice. Subsequently, plasma is collected by centrifugation of blood samples for 4 minutes at 14,000 rpm at room temperature, transferred to untreated microcentrifuge tubes, then frozen on dry ice and stored at −80° C. until analysis. Mice are sacrificed by decapitation, brains are rapidly micro-dissected into halves, flash frozen on dry ice and stored at −80° C. until analysis (one half for Abeta analysis and the other half for compound exposures measurement). For analysis of parenchymal A beta, brain samples are homogenized in 5.5 M guanidine-HCl buffer (0.5 mL per half brain) with tissue tearer (model 985-370) at speed 5 for about 1 minute. Homogenized brain samples are nutated overnight at room temperature.

For Abeta ELISA analysis, extracts are collected and diluted at least 1:10 in casein buffer (1× PBS with 0.25% casein, 0.05% Tween 20, 0.1% thimerosal, pH 7.4 with protease inhibitor cocktail (Sigma P9340 at 0.01 mg/mL)) and centrifuged at 14000 rpm for 10 minutes. For analysis of plasma A beta, samples are diluted 1:2 in specimen buffer (PBS; 0.05% Triton X-405; 0.04% thimerasol, 0.6% BSA), prior to analysis by ELISA. Plasma human A beta₁₋ₓ is determined by sandwich ELISA using m266.2 (anti-A beta₁₃₋₂₈) and biotinylated 3D6 (anti-A beta1-5) as the capture and reporter antibodies, respectively. Unknowns assayed in duplicate and pg/mL determined by interpolating (Soft Max Pro v. 5.0.1, Molecular Dynamics, using 4-parameter fit of the reference curve) from 8 point standard curves and then adjusting for dilution. Parenchymal Abeta is determined by sandwich ELISAs as described above and the values are normalized to protein levels (determined in duplicate by the Bradford Coomassie Plus Protein method) and expressed as pg/mg protein.

To determine the tissue and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### In Vivo Combination Study

In order to evaluate combinational plaque lowering therapy of an anti-N3pGlu Abeta antibody such as anti-N3pGlu Abeta antibody as described herein and a BACE inhibitor, such as a compound of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine, or a pharmaceutically acceptable salt thereof, a large cohort of PDAPP mice are first aged to 16 to 18-months of age. The aged PDAPP mice are randomized into five treatment arms based upon gender, parental line, and age. There are 20 to 30 aged PDAPP mice per treatment arm. Group 1 is sacrificed as a time zero at study initiation in order to determine the baseline level of pathology prior to therapeutic treatment (necropsy described below). The four remaining groups are then treated as follows: Group-2, control animals receiving placebo chow diet and weekly injections of 12.5 mg/kg of control isotype IgG2a antibody; Group-3, animals receiving weekly injections of 12.5 mg/kg anti-N3pGlu-Abeta antibody; Group-4, animals receiving BACE inhibitor chow diet at doses previously defined in the pilot feeding study, but typically ˜3 to 30 mg/kg/day; Group-5, animals receiving BACE inhibitor chow diet (˜3 to 30 mg/kg/day) and weekly injections of 12.5 mg/kg of anti-N3pGlu-Abeta antibody. The anti-N3pGlu-Abeta antibody is diluted from sterile stock solutions consisting of the antibody in PBS buffer and is administered to the animals by intraperitoneal injections. The BACE inhibitor is mixed with loose chow diet (˜0.15 to 1.5 mg compound per gram of feed depending upon desired dose) and compressed into feed pellets Animal weight is recorded at study initiation and subsequently weekly for the first month of treatment, and then monthly for the study duration. The food intake is also monitored over the course of the study at regular intervals. The animals receive the study treatments for a total of 4-months. The animals stay on their respective diets until necropsy, which occurs one week after the final antibody injections. At time of necropsy, the animals are anesthetized and blood obtained by cardiac puncture using EDTA (ethylenediaminetetraacetic acid) pre-rinsed 1 ml syringes. The blood samples are collected on ice and the plasma isolated by standard centrifugation. Subsequently, the animals are perfused with cold heparinized saline and the brain removed and dissected into the left and right hemi-spheres. One brain hemi-sphere is flash frozen and saved for histological analyses. The remaining brain hemi-sphere is dissected into tissue segments consisting of hippocampus, cortex, cerebellum, and mid-brain and subsequently frozen on dry ice. The plasma and tissue samples are stored at −80° C. until time of analysis.

### Pharmacokinetic Evaluation

Plasma pharmacokinetic is determined on the plasma samples obtained at time of necropsy. Plasma antibody levels are determined in an antigen binding ELISA assay (Herein “ELISA” refers to enzyme-linked immunosorbent assay) wherein plates are coated with antigen (A betaₚ₃₋₄₂) and subsequently incubated with diluted plasma samples or a reference standard consisting of a serial dilution of the anti-N3pGlu antibody in assay buffer (PBS+control murine plasma). After washing the plate, the bound murine antibody is detected with an anti-murine-HRP conjugated antibody followed by color development with TMB. To determine the tissue (mid-brain) and plasma levels of the BACE inhibitor, the following method is employed: A 0.1 mg/mL stock solution of BACE inhibitor is serially diluted with methanol/water (1:1, v/v), to prepare working solutions, which are then used to fortify control plasma and brain homogenates to yield analyte concentrations of 1, 5, 10, 20, 50, 100, 500, 1000, 2000, 4000, and 5000 ng/mL. Prior to analysis, brain samples are homogenized in 3-volumes of methanol/water (1:4, v/v) with an ultrasonic disrupter. An aliquot of each study sample, appropriate calibration standard and control matrix samples are transferred to a 96-well plate and then mixed with acetonitrile containing internal standard. After mixing, the samples are centrifuged to pellet the precipitated proteins. Aliquots of the resulting supernatants are then transferred to a clean 96-well plate and diluted with methanol/water (1:1, v/v), and 10 microliter aliquots are analyzed by LC-MS/MS. Analyte concentrations are calculated using the response to concentration relationship determined by multiple regression of the calibration curve samples.

### Pharmacodynamic Evaluation

The parenchymal Abeta concentrations are determined in guanidine solubilized tissue homogenates by sandwich ELISA. Tissue extraction is performed with the bead beater technology wherein frozen tissue is extracted in 1 ml of 5.5 M guanidine/50 mM Tris/0.5× protease inhibitor cocktail at pH 8.0 in 2 ml deep well dishes containing 1 ml of siliconized glass beads (sealed plates were shaken for two intervals of 3-minutes each). (“Tris” refers to tris(hydroxymethyl)aminomethane). The resulting tissue lysates are analyzed by sandwich ELISA for A beta₁₋₄₀and A beta₁₋₄₂: bead beater samples are diluted 1:10 in 2% BSA/PBS-T and filtered through sample filter plates (Millipore). (“PBS-T” refers to Phosphate Buffered Saline±Tween®.) Samples, blanks, standards, quality control samples, are further diluted in 0.55 M guanidine/5 mM Tris in 2% BSA/PBS-T prior to loading the sample plates. Reference standard are diluted in sample diluent. Plates coated with the capture antibody 21F12 (anti-A beta₄₂) or 2G3 (anti-A beta₄₀) at 15 μg/ml are incubated with samples and detection is accomplished with biotinylated 3D6 (anti-A beta₁₋ₓ) diluted in 2% BSA/PBS-T, followed by 1:20 K dilution NeutrAvidin-HRP (Pierce) in 2% BSA/PBS-T and color development with TMB (Pierce). The Abeta levels are interpolated from standard curves and the final tissue concentration is calculated as nanograms of Abeta per milligram of tissue wet weight. The percent area of the hippocampus and cortex occupied by deposited Abeta is determined histologically. Cryostat serial coronal sections (7 to 10 μm thick) are incubated with 10 μg/ml of biotinylated 3D6 (anti-A beta₁₋ₓ) or negative control murine IgG (biotinylated). Secondary HRP reagents specific for biotin are employed and the deposited Abeta visualized with DAB-Plus (DAKO). Immunoreactive Abeta deposits are quantified in defined areas of interest within the hippocampus or cortex by analyzing captured images with Image Pro plus software (Media Cybernetics).

These studies may show that the combination therapy of an anti-N3pGlu Abeta antibody and a BACE inhibitor, such as a compound of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine or a pharmaceutically acceptable salt thereof, may result in enhanced Abeta reductions relative to the individual mono-therapies.

Sequences

<SEQ ID NO: 1; PRT1; Artificial> HCDR1 - Antibody I and Antibody II
KASGYTFTDYYIN
<SEQ ID NO: 2; PRT1; Artificial> HCDR2 - Antibody I and Antibody II
WINPGSGNTKYNEKFKG
<SEQ ID NO: 3; PRT1; Artificial> HCDR3 - Antibody I and Antibody II
TREGETVY
<SEQ ID NO: 4; PRT1; Artificial> LCDR1 - Antibody I and Antibody II
KSSQSLLYSRGKTYLN
<SEQ ID NO: 5; PRT1; Artificial> LCDR2 - Antibody II
YAVSKLDS
<SEQ ID NO: 6; PRT1; Artificial> LCDR2 - Antibody I
YDVSKLDS
<SEQ ID NO: 7; PRT1; Artificial> LCDR3 - Antibody I and Antibody II
VQGTHYPFT
<SEQ ID NO: 8; PRT1; Artificial> HCVR - Antibody I and Antibody II
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCTREGETVYWGQ
GTLVTVSS
<SEQ ID NO: 9; PRT1; Artificial> LCVR - Antibody I
DVVMTQSPLSLPVTLGQPASISCKSSQSLLYSRGKTYLNWFQQRPGQSPRRLIYD
VSKLDSGVPDRFSGSGSGTDFTLKISRVEAEDVGVYYCVQGTHYPFTGQGTKLE
IK
<SEQ ID NO: 10; PRT1; Artificial> LCVR - Antibody II
DIQMTQSPSTLSASVGDRVTITCKSSQSLLYSRGKTYLNWLQQKPGKAPKLLIYA
VSKLDSGVPSRFSGSGSGTEFTLTISSLQPDDFATYYCVQGTHYPFTFGQGTKLEI
K
<SEQ ID NO: 11; PRT1; Artificial> Heavy Chain - Antibody I and Antibody II
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCTREGETVYWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
<SEQ ID NO: 12; PRT1; Artificial> Light Chain - Antibody I
DVVMTQSPLSLPVTLGQPASISCKSSQSLLYSRGKTYLNWFQQRPGQSPRRLIYD
VSKLDSGVPDRFSGSGSGTDFTLKISRVEAEDVGVYYCVQGTHYPFTFGQGTKLE
IKRTVAAPSVFIFPPSDEQLKSGTASVVCLLNNFYPREAKVQWKVDNA LQSGNSQ
ESVTEQDSKDSTYSLSSTLTLSKADYEKHKVYACEVTHQGLSSPVTKSFNRGEC
<SEQ ID NO: 13; PRT1; Artificial> Light Chain -Antibody 11
DIQMTQSPSTLSASVGDRVTITCKSSQSLLYSRGKTYLNWLQQKPGKAPKLLIYA
VSKLDSGVPSRFSGSGSGTEFTLTISSLQPDDFATYYCVQGTHYPFTFGQGTKLEI
KRTVAAPSVFIFPPSDEQLKSGTASVVCLLNNFYPREAKVQWKVDNA LQSGNSQ
ESVTEQDSKDSTYSLSSTLTLSKADYEKHKVYACEVTHQGLSSPVTKSFNRGEC
<SEQ ID NO: 14; DNA ; Artificial> Exemplified DNA for Expressing Antibody
Heavy Chain of SEQ ID NO: 11
CAGGTGCAGCTGGTGCAGTCTGGGGCTGAGGTGAAGAAGCCTGGGTCCTCGG
TGAAGGTCTCCTGCAAGGCTTCTGGATACACCTTCACCGACTATTATATCAAC
TGGGTGCGACAGGCCCCTGGACAAGGGCTTGAGTGGATGGGATGGATCAACC
CTGGCAGTGGTAATACAAAGTACAATGAGAAGTTCAAGGGCAGAGTCACGAT
TACCGCGGACGAATCCACGAGCACAGCCTACATGGAGCTGAGCAGCCTGAGA
TCTGAGGACACGGCCGTGTATTACTGTACAAGAGAAGGCGAGACGGTCTACT
GGGGCCAGGGAACCCTGGTCACCGTCTCCTCAGCCTCCACCAAGGGCCCATC
GGTCTTCCCGCTAGCACCCTCCTCCAAGAGCACCTCTGGGGGCACAGCGGCCC
TGGGCTGCCTGGTCAAGGACTACTTCCCCGAACCGGTGACGGTGTCGTGGAA
CTCAGGCGCCCIGACCAGCGGCGTGCACACCTTCCCGGCTGTCCTACAGTCCT
CAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGG
ACAAGAAAGTTGAGCCCAAATCTTGTGACAAAACTCACACATGCCCACCGTG
CCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGACGAGCTGACCAAGAACCAGGTCAGCCTGACCTGCCT
GGTCAAAGGCTTCTATCCCAGCGACATCGCCGTGGAGTGGGAGAGCAATGGG
CAGCCGGAGAACAACTACAAGACCACGCCCCCCGTGCTGGACTCCGACGGCT
CCTTCTTCCTCTATAGCAAGCTCACCGTGGACAAGAGCAGGTGGCAGCAGGG
GAACGTCTTCTCATGCTCCGTGATGCATGAGGCTCTGCACAACCACTACACGC
AGAAGAGCCTCTCCCTGTCTCCGGGT
<SEQ ID NO: 15; DNA ; Artificial> Exemplified DNA for Expressing Antibody
Light Chain of SEQ ID NO: 12
GATGTTGTGATGACTCAGTCTCCACTCTCCCTGCCCGTCACCCTTGGACAGCC
GGCCTCCATCTCCTGCAAGTCTAGTCAAAGCCTCCTGTACAGTCGCGGAAAAA
CCTACTTGAATTGGTTTCAGCAGAGGCCAGGCCAATCTCCAAGGCGCCTAATT
TATGATGTTTCTAAACTGGACTCTGGGGTCCCAGACAGATTCAGCGGCAGTGG
GTCAGGCACTGATTTCACACTGAAAATCAGCAGGGTGGAGGCTGAGGATGTT
GGGGTTTATTACTGCGTGCAAGGTACACACTACCCTTTCACTTTTGGCCAAGG
GACCAAGCTGGAGATCAAACGGACCGTGGCTGCACCATCTGTCTTCATCTTCC
CGCCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCCC
TCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTC
ACAAAGAGCTFCAACAGGGGAGACGTGC
<SEQ ID NO: 16; DNA ; Artificial> Exemplified DNA for Expressing Antibody
Light Chain of SEQ ID NO: 13
GACATCCAGATGACCCAGTCTCCTTCCACCCTGTCTGCATCTGTAGGAGACAG
AGTCACCATCACTTGCAAGTCCAGTCAGAGTCTCCTGTACAGTCGCGGAAAA
ACCTATTTGAACTGGCTCCAGCAGAAACCAGGGAAAGCCCCTAAGCTCCTGA
TCTATGCTGTCTCCAAACTGGACAGTGGGGTCCCATCAAGGTTCAGCGGCAGT
GGATCTGGGACAGAATTCACTCTCACCATCAGCAGCCTGCAGCCTGATGATTT
TGCAACTTATTACTGCGTGCAGGGTACACATTATCCTTTCACTTTTGGCCAGG
GGACCAAGCTGGAGATCAAACGGACCGTGGCTGCACCATCTGTCTTCATCTTC
CCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACA
GCACCTACAGCCTCAGCAGCACCCTGACGCTGAGCAAACTCAGACTACGAGAA
ACACAAAGTCTACGCCTGCGAAGTCACCCATCAGGGCCTGAGCTCGCCCGTC
ACAAAGAGCTTCAACAGGGGAGAGTGC
<SEQ ID NO: 17; PRT1; Artificial> (LCDR1 - B12L/R17L/hE8L)
KSSQSLLYSRGKTYLN
<SEQ ID NO: 18; PRT1; Artificial> (LCDR2 - B12L/R17L/hE8L)
AVSKLDS
<SEQ ID NO: 19; PRT1; Artificial> (LCDR3 - B12L/R17L/hE8L)
VQGTHYPFT
<SEQ ID NO; 20; PRT1; Artificial> (HCDR1 - B12L)
GYDFTRYYIN
<SEQ ID NO: 21; PRT1; Artificial> (HCDR1 - R17L)
GYTFTRYYIN
<SEQ ID NO: 22; PRT1; Artificial> (HCDR2 - B12L/R17L/hE8L)
WINPGSGNTKYNEKFKG
<SEQ ID NO: 23; PRT1; Artificial> (HCDR3 - B12L)
EGITVY
<SEQ ID NO: 24; PRT1; Artificial> (HCDR3 - R17L)
EGTTVY
<SEQ ID NO: 25; PRT1; Artificial> (LCVR - B12L/R17L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
<SEQ ID NO: 26; PRT1; Artificial> (HCVR - B12L)
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSS
<SEQ ID NO: 27; PRT1; Artificial> (HCVR - R17L)
QVQLVQSGAEVKKPGSSVKVSCKASGYTFTRYYINWVRQAPGQGLEWMGWINP
GSGNTKYNEKFKGRVTITADESTSTAYMELSSLRSEDTAVYYCAREGTTVYWCQ
GTTVTVSS
<SEQ ID NO: 28; PRT1; Artificial> (LC - B12L/R17L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
<SEQ ID NO: 29; PRT1; Artificial> (HC - B12L)
QVQLVQSGAEVKKPGSSVKVSCKASGYDFTRYYINWVRQAPGQGLEWMGWINP
GSGNTKYNEKFKGRVTITFADESTSTAYMELSSLRSEDTAVYYCAREGITVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
<SEQ ID NO: 30; PRT1; Artificial> (HC - R17L)
QVQLVQSGAEVKKPGSSVKVSCKASGYTFTRYYINWVRQAPGQGLEWMGWINP
GSGNTKYNEKFKGRVTITADESTSTAYMELSSLRSEDTAVYYCAREGTTVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
N3pGlu Aβ (SEQ ID NO: 31)
[pE]FRHDSGYEVHHQKLVFFAEDVGSNKGAIIGLMVGGVVIA
<SEQ ID NO, 32; PRT1; Artificial> (LCVR-hE8L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
<SEQ ID NO, 33; PRT1; Artificial> (LC-hE8L)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVHFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
<SEQ ID NO, 34; PRT1; Artificial> (HCVR-hE8L)
QVQLVQSGAEVKKPGSSVKVSCKASGYTFTDYYINWVRQAPGQGLEWMGWINP
GSGNTKYNEKFKGRVTITADESTSTAYMELSSLRSEDTAVYYCAREGETVYWGQ
GTTVTVSS
<SEQ ID NO, 35; PRT1; Artificial> (HC-hE8L)
QYQLVQSGAEVKKPGSSVKVSCKASGYTFTDYYINWVRQAPGQGLEWMGWINP
GSGNTKYNEKFKGRVTITADESTSTAYMELSSLRSEDTAVYYCAREGETVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
<SEQ, ID NO: 36; PRT1; Artificial> (HCDR1-hE8L)
GYTFTDYYIN
<SEQ ID NO: 37; PRT1; Artificial> (HCDR3-hE8L)
EGETVY
<SEQ ID NO: 38; PRT1; Artificial> (Aβ 1-42)
DAEFRHDSGYEVHHQKLVFFAEDVGSNKGAIIGLMVGGVVIA

1. A method of treating a patient having a disease characterized by deposition of Aβ, comprising administering to a patient in need of such treatment an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine or a pharmaceutically acceptable salt thereof, in combination with an effective amount of an anti-N3pGlu Abeta antibody.

2. The method according to claim 1 wherein the compound is a camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine.

3. The method according to claim 1, wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

4. The method according to claim 1, wherein the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

5. The method according to claim 1, wherein the anti-N3pGlu Abeta antibody comprises a light chain (LC) and a heavy chain (HC), wherein said LC and HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

6. The method according to claim 1, wherein the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

7. The method according to claim 1, wherein the disease characterized by deposition of Aβ is selected from a group consisting of clinical or pre-clinical Alzheimer's disease (AD), Down's syndrome, and clinical or pre-clinical cerebral amyloid angiopathy, prodromal AD, mild AD, moderate AD and severe AD.

8-9. (canceled)

10. The method according to according to claim 1, wherein:
the compound is administered prior to the administration of the anti-N3pGlu Abeta antibody;
the anti-N3pGlu Abeta antibody is administered prior to the administration of the compound; or
the compound and the anti-N3pGlu Abeta antibody are administered simultaneously.

11-17. (canceled)

18. A pharmaceutical composition, comprising a compound (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine or a pharmaceutically acceptable salt thereof, with one or more pharmaceutically acceptable carriers, diluents, or excipients, in combination with a pharmaceutical composition of anti-N3pGlu Abeta antibody, with one or more pharmaceutically acceptable carriers, diluents, or excipients.

19. The pharmaceutical composition according to claim 18 wherein the compound is a camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine.

20. The pharmaceutical composition according to claims 18, wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

21. The pharmaceutical composition according to claim 18, wherein the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

22. The pharmaceutical composition according to claim 18, wherein the anti-N3pGlu Abeta antibody comprises a light chain (LC) and a heavy chain (HC), wherein said LC and HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

23. The pharmaceutical composition according to claim 18, wherein the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

24. A kit for treatment of Alzheimer's disease, wherein the kit comprises an effective amount of a compound which is (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine or a pharmaceutically acceptable salt thereof, and an effective amount of an anti-N3pGlu Abeta antibody.

25. The kit according to claim 24 wherein the compound is a camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine.

26. The kit according to claim 24, wherein the anti-N3pGlu Abeta antibody comprises a light chain variable region (LCVR) and a heavy chain variable region (HCVR), wherein said LCVR comprises LCDR1, LCDR2 and LCDR3 and HCVR comprises HCDR1, HCDR2 and HCDR3 which are selected from the group consisting of:
a) LCDR1 is SEQ ID. NO: 17, LCDR2 is SEQ ID. NO: 18, LCDR3 is SEQ ID. NO: 19, HCDR1 is SEQ ID. NO: 20, HCDR2 is SEQ ID: NO: 22, and HCDR3 is SEQ ID. NO: 23; and
b) LCDR1 is SEQ ID. NO: 17, LCDR2 is SEQ ID. NO: 18, LCDR3 is SEQ ID. NO: 19, HCDR1 is SEQ ID. NO: 21, HCDR2 is SEQ ID. NO: 22, and HCDR3 is SEQ ID. NO: 24;
c) LCDR1 is SEQ ID. NO: 17, LCDR2 is SEQ ID. NO: 18, LCDR3 is SEQ ID. NO: 19, HCDR1 is SEQ ID. NO: 36, HCDR2 is SEQ ID. NO: 22, and HCDR3 is SEQ ID. NO: 37;
d) LCDR1 is SEQ ID. NO: 4, LCDR2 is SEQ ID. NO: 6, LCDR3 is SEQ ID. NO: 7, HCDR1 is SEQ ID. NO: 1, HCDR2 is SEQ ID. NO: 2, and HCDR3 is SEQ ID. NO: 3;
e) LCDR1 is SEQ ID. NO: 4, LCDR2 is SEQ ID. NO: 5, LCDR3 is SEQ ID. NO: 7, HCDR1 is SEQ ID. NO: 1, HCDR2 is SEQ ID. NO: 2, and HCDR3 is SEQ ID. NO: 3.

27. The kit according to claim 24, wherein the anti-N3pGlu Abeta antibody comprises a light chain variable region (LCVR) and a heavy chain variable region (HCVR), wherein said LCVR and HCVR are selected from the group consisting of
a) LCVR of SEQ ID NO: 25 and HCVR of SEQ ID NO: 26;
b) LCVR of SEQ ID NO: 25 and HCVR of SEQ ID NO: 27;
c) LCVR of SEQ ID NO: 32 and HCVR of SEQ ID NO: 34;
d) LCVR of SEQ ID NO: 9 and HCVR of SEQ ID NO: 8; and
e) LCVR of SEQ ID NO: 10 and HCVR of SEQ ID NO: 8.

28. The kit according to claim 24, wherein the anti-N3pGlu Abeta antibody comprises a light chain (LC) and a heavy chain (HC), wherein said LC and HC are selected from the group consisting of
f) LC of SEQ ID NO: 28 and HC of SEQ ID NO: 29;
g) LC of SEQ ID NO: 28 and HC of SEQ ID NO: 30;
h) LC of SEQ ID NO: 33 and HC of SEQ ID NO: 35;
i) LC of SEQ ID NO: 12 and HC of SEQ ID NO: 11; and
j) LC of SEQ ID NO: 13 and HC of SEQ ID NO: 11.

29. The kit according to claim 24, wherein the anti-N3pGlu Abeta antibody comprises two light chains (LC) and two heavy chains (HC), wherein each LC and each HC are selected from the group consisting of
a) LC of SEQ ID NO: 28 and HC of SEQ ID NO: 29;
b) LC of SEQ ID NO: 28 and HC of SEQ ID NO: 30;
c) LC of SEQ ID NO: 33 and HC of SEQ ID NO: 35;
d) LC of SEQ ID NO: 12 and HC of SEQ ID NO: 11; and
e) LC of SEQ ID NO: 13 and HC of SEQ ID NO: 11.

30-38. (canceled)
